(19) United States
(12) Patent Application Publication
(10) Pub. No.: US 2024/0068682 A1
(43) Pub. Date: Feb. 29, 2024

(54) SYSTEMS AND METHODS FOR THERMAL INACTIVATION OF PATHOGENS

(71) Applicant: RESEARCH PRODUCTS CORPORATION, Madison, WI (US)

(72) Inventors: Guolian Wu, Madison, WI (US); Michelle Harasimowicz, Madison, WI (US); Andrew Litch, Madison, WI (US); Thomas J. Anoszko, Madison, WI (US)

(73) Assignee: RESEARCH PRODUCTS CORPORATION, Madison, WI (US)

(21) Appl. No.: 18/240,164

(22) Filed: Aug. 30, 2023

Related U.S. Application Data
(60) Provisional application No. 63/402,691, filed on Aug. 31, 2022.

(51) Int. Cl. F24F 8/20; A61L 9/18; F24F 8/108

(52) U.S. Cl. CPC F24F 8/20; A61L 9/18; F24F 8/108; A61L 2209/14

(57) ABSTRACT

A system and method for controlling air quality within an indoor space are disclosed. An example system includes an air circulation unit that moves air through ductwork of a heating, ventilation, and air conditioning (HVAC) system and an air sanitization unit within the ductwork of the HVAC system that sanitizes air passing through the ductwork of the HVAC system. The system further includes an indoor air quality controller that controls a rate at which the air circulation unit moves the air through the ductwork of the HVAC system responsive to inputs received at the indoor air quality controller and controls an operational status of the air sanitization unit responsive to the inputs received at the indoor air quality controller.

### CROSS-REFERENCE TO RELATED APPLICATIONS

The present application claims priority to U.S. Provisional Patent Application No. 63/402,691, filed Aug. 31, 2022, which is incorporated by reference herein in its entirety.

### BACKGROUND

The present disclosure relates generally to systems and methods for sanitizing air within a structure. More specifically, the disclosure relates to systems and methods for thermal inactivation of microbes and pathogens in heating, ventilation, and air conditioning (HVAC) systems and air filtration systems.

HVAC systems are designed to regulate characteristics of indoor air to provide a comfortable and safe living environment within a structure. Such characteristics may include temperature, humidity, and a variety of air quality parameters. Current HVAC systems may incorporate one or more of a variety of components for regulating such characteristics including a furnace or other heating component, an air condition system or other cooling component, ventilation components, humidifiers, dehumidifiers, etc. Discussed in the present disclosure are various improved systems and methods for improving air quality and/or sanitizing air in coordination with other components and aspects of HVAC systems.

### SUMMARY

According to one aspect of the present disclosure, a system for thermal inactivation of pathogens includes an air duct configured to deliver air to an indoor space, an air filter disposed within the air duct, a heat element disposed within the air duct proximate the air filter, and a separator element disposed within the air duct proximate the heat element. The heat element is configured to heat the air filter and maintain the air filter at a threshold temperature for a microbial inactivation period. The separator element is configured to at least partially isolate the heat element and the air filter from an upstream end or a downstream end of the air duct.

According to another aspect of the present disclosure, a system for thermal inactivation of pathogens includes an air duct configured to delivery air to an indoor space, an air filter disposed within the air duct, and a heat element disposed within the air duct and engaging the air filter. The heat element is operable to selectively control a temperature of the air filter and maintain the air filter at a threshold temperature for a microbial inactivation period.

According to another aspect of the present disclosure, a system for thermal inactivation of pathogens includes a microbial inactivation control unit, a heat element, and a separator element. The heat element and the separator element are both communicably coupled to the microbial inactivation control unit. The microbial inactivation control unit includes a communications interface that is configured to receive data from at least one of an air condition sensor or a heating, ventilation, and air conditioning (HVAC) control unit. The separator element is operable to selectively insulate the heat element from an environment surrounding the heat element. The microbial inactivation control unit is configured to coordinate operation of the heat element and the separator element in response to the data indicating that the HVAC system is in an idle state.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

A clear conception of the advantages and features constituting the present disclosure, and of the construction and operation of typical mechanisms provided with the present disclosure, will become more readily apparent by referring to the exemplary, and therefore non-limiting, embodiments illustrated in the drawings accompanying and forming a part of this specification, wherein like reference numerals designate the same elements in the several views, and in which:

FIG. 1 is a block diagram of an HVAC system, according to an illustrative embodiment.

FIG. 2 is a side cross-sectional view of a thermal microbial inactivation system (TMIS) in a first state of operation, according to an illustrative embodiment.

FIG. 3 is a side cross-sectional view of the TMIS of FIG. 2 in a second state of operation.

FIG. 4 is a side cross-sectional view of a TMIS in a first state of operation, according to another illustrative embodiment.

FIG. 5 is a side cross-sectional view of the TMIS of FIG. 4 in a second state of operation.

FIG. 6 is a side cross-sectional view of a TMIS in a first state of operation, according to another illustrative embodiment.

FIG. 7 is a side cross-sectional view of the TMIS of FIG. 6 in a second state of operation.

FIG. 8 is a side cross-sectional view of a TMIS in a first state of operation, according to another illustrative embodiment.

FIG. 9 is a side cross-sectional view of the TMIS of FIG. 8 in a second state of operation.

FIG. 10 is a side cross-sectional view of a TMIS in a first state of operation, according to another illustrative embodiment.

FIG. 11 is a side cross-sectional view of the TMIS of FIG. 10 in a second state of operation.

FIG. 12 is a side cross-sectional view of a TMIS, according to another illustrative embodiment.

FIG. 13 is a side cross-sectional view of a TMIS, according to another illustrative embodiment.

FIG. 14 is a front view of an air filter element of the TMIS of FIG. 13.

FIG. 15 is a flow diagram of a method of controlling a TMIS, according to an illustrative embodiment.

The foregoing and other features of the present disclosure will become apparent from the following description and appended claims, taken in conjunction with the accompanying drawings. Understanding that these drawings depict only several embodiments in accordance with the disclosure and are therefore, not to be considered limiting of its scope, the disclosure will be described with additional specificity and detail through use of the accompanying drawings.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

Heating, ventilation, and air conditioning (HVAC) systems may be configured to provide treated air to different parts of a structure (e.g., an indoor space, a building, a residence, etc.). The HVAC system may include ductwork to carry treated air throughout the structure. Such HVAC systems often include one or more of a furnace, air conditioner, humidifier, dehumidifier, etc., to heat, cool, modify a humidity level, or otherwise treat the air. The HVAC system may also include an air filtration system to remove dirt, pollen, and other particulates from the air before reintroducing the treated air into the structure. The air filtration system may also capture microbes and other pathogens from the air.

Referring generally to the figures, improved microbial inactivation systems for use with an HVAC system (e.g., a residential HVAC system, a commercial or industrial HVAC system, etc.) or a standalone air filtration system are disclosed. The microbial inactivation systems provide inactivation of microbes and other pathogens that can be captured on an air filtration system, to prevent accumulation and multiplication of any microorganisms, which could otherwise shed or leach off from the air filtration system and pass into the treated air. The microbial inactivation systems of the present disclosure may be incorporated into the HVAC system, into the ductwork used for the air filtration system, without requiring separate treatment passages or sections of ductwork. The microbial inactivation systems of the present disclosure may also include a control unit that is configured to coordinate operation of the indoor air quality system with the HVAC system so as to improve the effectiveness of microbial inactivation and overall operational efficiency.

) In contrast with existing systems for antimicrobial control such as UV-light based systems, the microbial inactivation systems of the present disclosure are configured to substantially eliminate viruses, bacteria, and other microbes and pathogens by heating an air filter element within the air filtration system to increase the temperature of the air filter element to levels and for time periods that are unsuitable for microbial survival and growth. The microbial inactivation systems of the present disclosure are configured to facilitate even/uniform distribution of heat across the cross-section of the ductwork and/or area of purification (e.g., an area proximal the air filter, etc.) to ensure more complete air purification. In at least one aspect, the microbial inactivation system is configured to partially isolate the air filter element within an inner chamber of the ductwork during heating, which can ensure heat is fully/uniformly distributed across the entire air filter and can inactivate microbes within crevices and other hard to access regions of the air filtration system. Isolating the air filter element during treatment can also improve overall system efficiency by reducing heat loss and increasing heat transfer to the air filter element.

In another aspect, the microbial inactivation system is configured to irradiate both an inlet side (e.g., an upstream side, etc.) and an outlet side (e.g., a downstream side, etc.) of the filtration system (e.g., an air filter, an air purification area, etc.), providing complete coverage over spaces within the ductwork and across air filter in which pathogens may be captured or retained. For example, the microbial inactivation system may include infrared heating elements on both sides of the air filter and/or air purification area (e.g., air purification passage, inner chamber, etc.) that are arranged to expose or otherwise cover all of the surfaces of the air filter to infrared light. In another aspect, the microbial inactivation system is configured to heat the air filter by placing heating panels in direct contact with surfaces on either side of the air filter (e.g., the upstream side and the downstream side) to provide heating to all exposed sides and/or surfaces of the air filter.

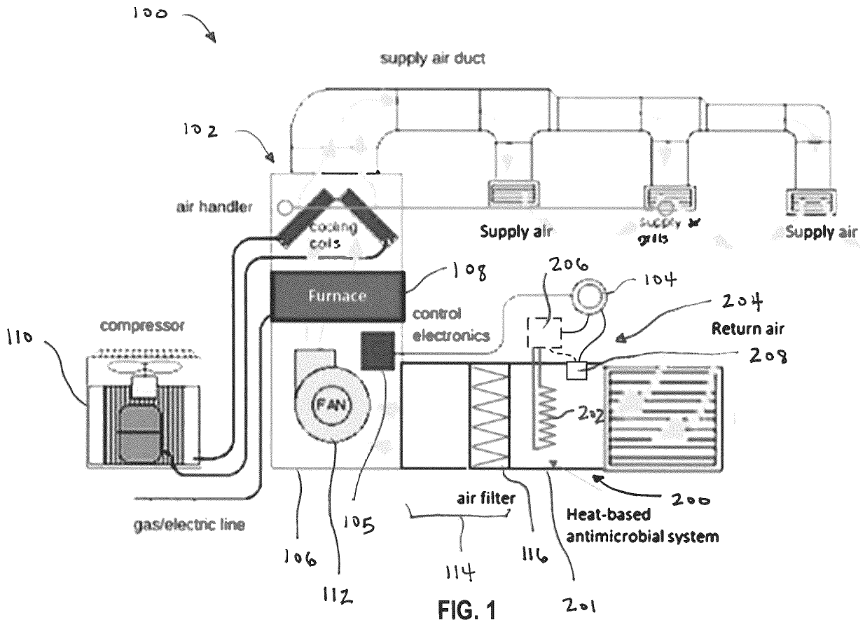

Referring to FIG. 1, a schematic representation of an air quality system 100 is shown, according to an illustrative embodiment. As shown, the air quality system 100 includes an HVAC system 102 that is configured to treat incoming air and deliver the treated air to different spaces within a structure. The air quality system 100 includes an indoor air quality (IAQ) controller 104 configured to control one or more components of an HVAC system.

The IAQ controller 104 may be an in-home control device, a remote control device such as a server device located on a cloud or as an app, or any other control device known to those in the art. The IAQ controller 104 may also be implemented in combination with other control devices (e.g., a control device that jointly controls multiple HVAC components including temperature, humidity, ventilation, etc.). For example, the IAQ controller 104 may be or form part of an HVAC control unit (e.g., thermostat, etc.) that is configured to control HVAC equipment based on user inputs and/or sensor data received at the thermostat.

As shown in FIG. 1, the IAQ controller 104 may be an HVAC control unit is communicably coupled (e.g., electrically connected, wired or wirelessly connected, etc.), via at least one control module 105 (e.g., solenoid, control circuit, etc.), to an HVAC component that includes an air circulation unit configured to selectively move air through a ductwork 106 (e.g., conduit, air duct, etc.). The HVAC component may be a furnace 108, air conditioning unit 110, an air circulation unit 112, or other control unit designed to effect a characteristics of air within an indoor space. The air circulation unit 112 may be a blower, fan, or other component configured to selectively move air through the ductwork 106. The HVAC control unit may be configured to transmit commands to the HVAC component and to receive data regarding an operational status of the HVAC component. In at least one embodiment, the HVAC controller is communicably coupled to other remote computing devices (e.g., other control units, etc.) and is configured to transmit data indicating an operational status of the HVAC system (e.g., whether the HVAC system is in an idle state, which components have been activated, a position of a ventilation damper, etc.).

It should be appreciated that the HVAC system 102 may include additional, fewer, and/or different components or equipment in other illustrative embodiments. For example, the IAQ controller 104 may also be communicably coupled to a humidifier to selectively increase humidity of the air within the ductwork and a dehumidifier to selectively decrease humidity within the indoor space. The IAQ controller 104 may be further communicably coupled to and configured to control at least one ventilation damper or power ventilator to selectively provide outside air into the HVAC system 102 and at least one zone damper to selectively and independently control air quality within one or more zones (e.g., rooms, spaces, etc.) of the structure.

As shown in FIG. 1, the HVAC system 102 may also include an air filtration system 114 that is configured to remove dirt, pollen, microbes, and other particulate from the incoming air. The air filtration system 114 may include an air filter element, shown as air filter 116. The air filter 116 may include a filter media (e.g., a pleated and/or corrugated filter media, etc.) that captures incoming particulate. The air filter element 116 may be a high efficiency particulate air (HEPA) air filter configured to remove approximately 99.97% of dust, pollen, mold, bacteria, and other airborne particles with a size of 0.3 microns, or another suitable air filter type.

The air quality system 100 may further include a thermal microbial inactivation system (TMIS) 200 in FIG. 1. In at least one embodiment, the air filtration system 114 may form part of the TMIS 200 so that the TMIS 200 may be manufactured with the air filtration system 114 as a single unit. For example, the TMIS 200 may include an air duct 201 (e.g., a conduit, etc.) that is configured to receive the air filter 116 and to couple the air filter to the HVAC system 102. In other embodiments, the TMIS may be retrofit into an existing air filtration system (e.g., a standalone air filtration system, an air filtration system for an HVAC system, etc.).

The TMIS 200 may include further include a heat element 202, a separator element (not shown), and a microbial inactivation control system 204 that is communicably coupled to the heat element 202 and the separator element, and that is configured to coordinate operation of the heat element 202 and the separator element (e.g., based on an operating state of the HVAC system 102, sensor data, etc.). The microbial inactivation control system 204 may include a microbial inactivation control unit 206 (e.g., a microbial inactivation controller, control circuit, module, etc.) and at least one air condition sensor 208 that is configured to provide an environmental condition (e.g., a flow rate, a temperature, etc.) associated with the air in the air duct. The microbial inactivation control unit 206 may be communicably coupled to the IAQ controller 104 and/or the at least one air condition sensor 208. For example, the microbial inactivation control unit 206 may include a communications interface that is configured to receive data from at least one of the air condition sensor or the IAQ controller 104 (e.g., the HVAC control unit). In one embodiment, the microbial inactivation control unit 206 is a standalone control circuit that is separate from the IAQ controller 104. In another embodiment, the microbial inactivation control unit 206 forms part of the IAQ controller 104 (e.g., as a control circuit or module within the IAQ controller 104, etc.).

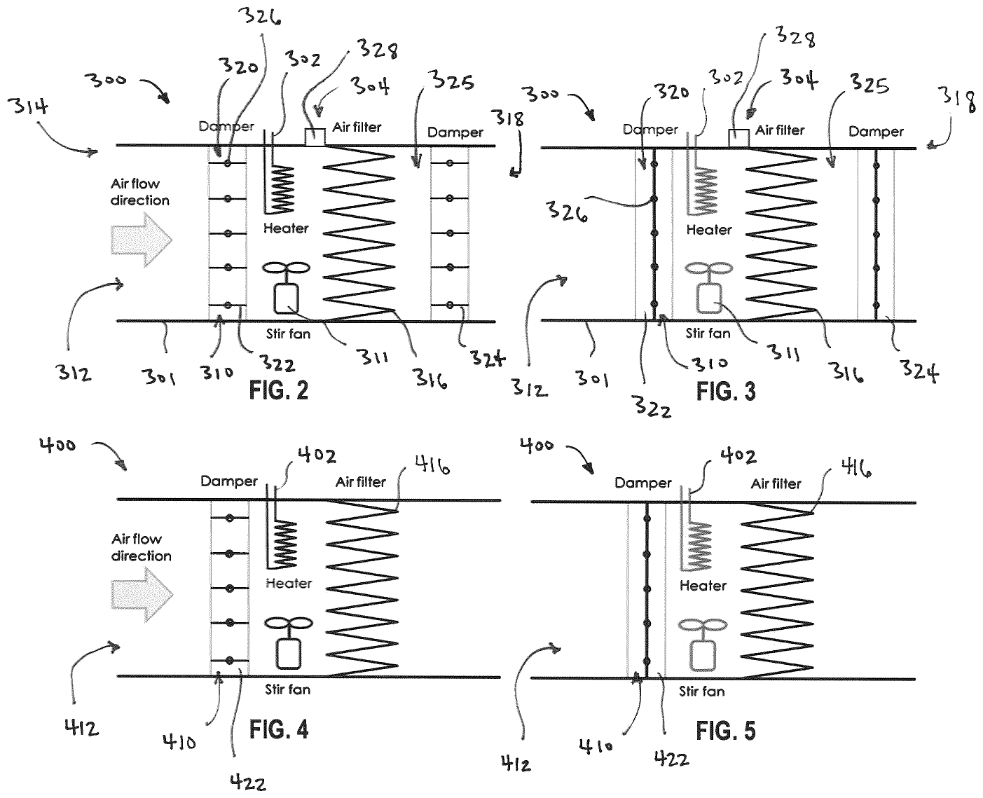

FIG. 2 and FIG. 3 show a TMIS 300 for an HVAC system or a standalone air filtration system, according to an illustrative embodiment. The TMIS 300 includes an air duct 301 (e.g., an air duct, a conduit, etc.), an air filter element (shown as air filter 316), a heat element 302, a separator element 310, an air driver 311, and a microbial inactivation control system 304. In other embodiments, the TMIS 300 may include additional, fewer, and/or different components.

The air duct 301 is configured to deliver air to an indoor space. The air duct 301 may include walls defining an enclosed channel or air passage 312. The air duct 301 may be configured to direct air along the air passage 312 between an upstream end 314 and a downstream end 318. The upstream end 314 may be configured as an air return that is configured to receive air (e.g., untreated air, dirty air, etc.) from an indoor space or from an environment surrounding a structure. The air duct 301 may be configured to couple to an HVAC system at the downstream end 318, to provide clean, filtered air to the HVAC system. In another embodiment, the air duct 301 may be configured to provide clean, filtered air directly to an indoor space.

The air filter element, shown as air filter 316 is disposed within the air duct 301 at an intermediate position along the air duct 301 between the upstream end 314 and the downstream end 318. The air filter 316 may be a replaceable air filter panel that is sealingly engaged with the air duct 301 and extends across an entire span of the air passage 312 so as to prevent bypass of untreated air across the air filter 316. In at least one embodiment, the TMIS 300 further includes a mount that is coupled to the air duct 301 and that is configured to detachably couple and sealingly engage the air filter 316 with the air duct 301.

The TMIS 300 is configured to provide an even distribution of heat across the cross-section of the air duct 301 and/or area of purification (e.g., treatment area, inner chamber within which the air filter 316 is positioned, etc.). In at least one embodiment, multiple heat elements are positioned within the air duct 301 to fully irradiate both sides of the air filter 316 and/or area of purification. In other embodiments, a separator element is used to at least partially isolate the area of purification (e.g., the air filter 316, the heat element 302, etc.) and to facilitate heat transfer to the entire air filter.

The heat element 302 (e.g., heat source, heater, etc.) is configured to heat the air filter 316 and maintain the air filter 316 at a threshold temperature for a microbial inactivation time period, as will be further described. The heat element 302 may be disposed in the air duct 301 proximate the air filter 316, between the air filter 316 and an upstream end 314 or downstream end 318 of the air filter. In some embodiments, the heat element 302 may be one of multiple heat elements. For example, the TMIS 300 may include a heat element disposed on each side of the air filter 3016 (e.g., an upstream side and a downstream side of the air filter 316). The heat elements may be positioned within the air duct 301 to transfer heat directly to all of the exposed surfaces of the air filter 316. The heat element 302 may be a nichrome wire heater (e.g., made from a nichrome-60 wire, etc.), an infra-red heating element, a resistive heater, or another suitable heater type. The heat element 302 may be directed toward the heater (e.g., facing the heater) and may be disposed at a central position within the air passage 312 to provide uniform coverage over the air filter 316. In at least one embodiment, the heat element 302 is sized and positioned to irradiate or otherwise provide heat to an entire surface of the air filter 316.

The separator element 310 is operable to selectively isolate the heat element 302 and/or air filter 316 from an environment surrounding the heat element 302 and/or air filter 316 so as to reduce heat loss to the environment, facilitate uniform heating of the air filter 316 and/or area of purification, and increase the overall rate of heat transfer from the heat element 302 to the air filter 316 and/or area of purification. In the embodiment of FIG. 2 and FIG. 3, the separator element 310 is configured to at least partially isolate a section of the air duct 301 containing the heat element 302 and the air filter 316, and to at least partially block air flow from the upstream end 314 and/or the downstream end 318 of the air duct 301 so as to reduce heat loss to air entering the air duct 301 and passing therethrough. The separator element 310 also defines an area of purification within which the heated air can distribute.

In the embodiment of FIG. 2 and FIG. 3, the separator element 310 is disposed within the air duct 301 proximate the heat element 102. The separator element 310 may be operable (e.g., in response to commands from the microbial inactivation control system 304) between a first operating mode (shown in FIG. 2) in which the separator element 310 allows air to flow therethrough along the air passage 312 between the upstream end 314 and the downstream end 318 to a second operating mode (shown in FIG. 3) in which the separator element 310 at least partially blocks air from flowing between the upstream end 314 and the downstream end 318. In other embodiments, the separator element 310 may be another type of actuatable and/or movable device that is configured to insulate the heat element 302 and/or air filter 316.

As shown in FIG. 2 and FIG. 3, the separator element 310 may include a damper assembly 320 including a first damper 322, a second damper 324, and at least one damper actuator 326. The first damper 322 and the second damper 324 may be disposed on opposing sides of the air filter 316. Together, the first damper 322 and the second damper 324 define an inner chamber 325 (e.g., an area of purification, a microbial inactivation chamber, an air treatment chamber, etc.) containing the air filter 316. The first damper 322 may be operable to at least partially block airflow from passing between the upstream end 314 and the air filter 316. The second damper 324 may be operable to at least partially block airflow from passing between the air filter 316 and the downstream end 318. The first damper 322 and the second damper 324 may include a plurality of vanes, blades, etc. extending across the air passage 312 that are rotatable to control a flow rate through the inner chamber 325 and to at least partially isolate the inner chamber 325, which can provide uniform heating of the air filter 316 and a more even distribution of heat across the cross-section of the air duct 301 (e.g., which can minimize temperature gradients within the inner chamber 325, etc.).

The damper actuator 326 is operably coupled to the first damper 322 and/or the second damper 324 and is configured to reposition the damper(s) between an open position (corresponding with the first mode of operation as shown in FIG. 2) and a closed position (corresponding with the second mode of operation as shown in FIG. 3). In at least one embodiment, the damper actuator 326 is communicably coupled and/or forms part of the microbial inactivation control system 304 and is configured to receive commands from the microbial inactivation control system 304 to reposition the first damper 322 and the second damper 324 (e.g., in response to a command to perform germicidal treatment of the air filter 316).

The air driver 311 is configured to circulate air within a space between the heat element 302 and the air filter 316 when the separator element 310 is in the second mode of operation to increase heat transfer between the heat element 302 and the air filter 316 (e.g., to increase convective heat transfer between the heat element 302 and the air filter 316). In embodiments in which the heat element 302 is located on only one side of the air filter 316, the air driver 311 can cause heated air to pass through the air filter 316 (e.g., through the filter media) to provide a more uniform distribution of heat across the air filter 316 (to heat both sides of the air filter 316) and to facilitate heat transfer to all areas of the inner chamber 325. The air driver 311 can also recirculate hot air within the inner chamber 325, which can minimize temperature gradients within the inner chamber 325. The air driver 311 may be a fan (e.g., a stir fan, blower, etc.), or another suitable actuator that is configured to stir or otherwise agitate the air within the inner chamber 325 (e.g., between the heat element 302 and the air filter 316, across the air filter 316, etc.).

The microbial inactivation control system 304 is communicably coupled to the heat element 302, the separator element 310, and the air driver 311 and is configured to control operation of the heat element 302, the separator element 310, and the air driver 311. In at least one embodiment, the microbial inactivation control system includes a microbial inactivation control unit (see FIG. 1) and at least one sensor communicably coupled (e.g., via wired or wireless connection, etc.) to the microbial inactivation control unit.

For example, the microbial inactivation control system 304 may include an air condition sensor 328 disposed at least partially within the air passage 312. In one embodiment, the air condition sensor 328 may be configured to monitor (e.g., measure, determine, etc.) a temperature and/or flow rate of air passing through the air passage 312. In another embodiment, the air condition sensor 328 may be configured to monitor a temperature or condition of the air filter 316 (e.g., a temperature of the filter media, a temperature proximate the filter media, a temperature of the air within the inner chamber 325, etc.). In such an embodiment, the air condition sensor 328 may be positioned in direct contact with (e.g., engaged, etc.) with the air filter 316 (e.g., the filter media, etc.). The microbial inactivation control unit may be configured to coordinate operation of the heat element 302, the separator element 310, and/or the air driver 311 in response to data from the air condition sensor 328 and/or data from the IAQ controller, as will be further described.

The microbial inactivation system arrangement described with reference to FIG. 2 and FIG. 3 should not be considered limiting. It should be appreciated that various modifications and alterations are possible without departing from the inventive principles disclosed herein. For example, FIG. 4 and FIG. 5 show another example embodiment of a TMIS 400 in which the separator element 410 includes only a single damper 422 to block flow through the air passage 412. The damper 422 is disposed upstream of the air filter 416 and defines an enclosed space that contains the heat element 402 (e.g., an enclosed space between the damper 422 and the air filter 416, etc.). In other embodiments, the damper may be positioned downstream of the air filter 416 to selectively block air flow through the air passage 412.

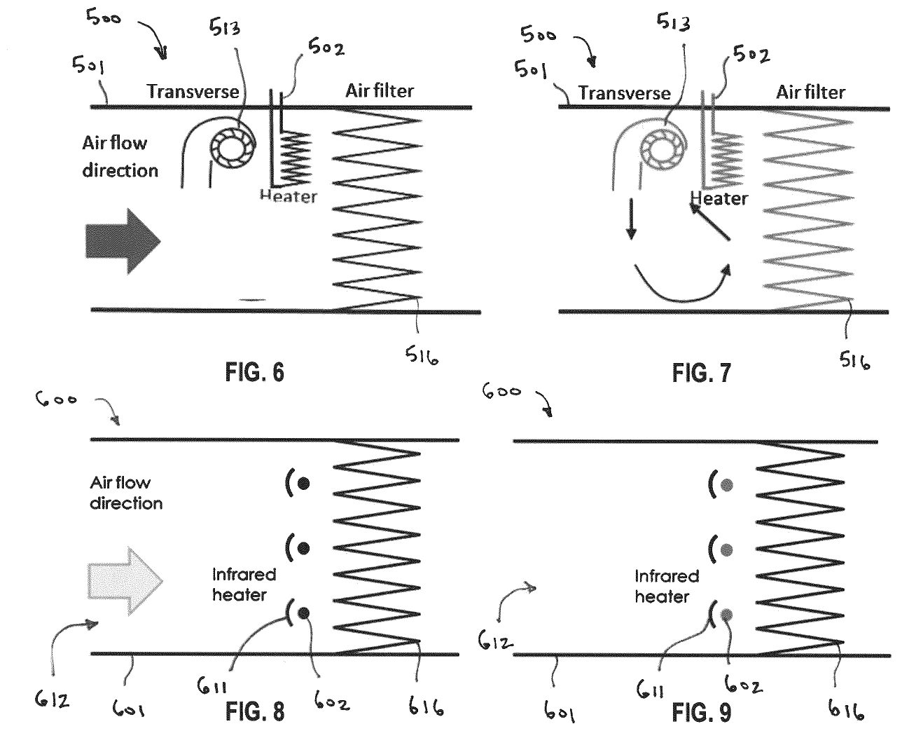

In other embodiments, the damper may be replaced with another suitable air blocking device or air flow mitigation device. For example, FIG. 6 and FIG. 7 show an example embodiment of a TMIS 500 that in which the separator element and the air driver comprise a single transverse air driver 513. The transverse air driver 513 is coupled to the air duct 501 along an upper wall of the air duct 501. The transverse air driver 513 may be positioned at least partially within the air passage 512 or may be positioned outside of the air passage 512 and direct air into the air passage 512 through an opening in the outer wall of the air duct 501.

As shown in FIG. 7, when activated (e.g., in the second mode of operation, etc.), the transverse air driver 513 is configured to is configured to produce an air curtain proximate the heat element 502 and/or air filter 516 that extends across the air passage 512 (e.g., laterally, from side-to-side, etc.). The air curtain at least partially isolates the heat element 502 and air filter 516 from air flow through the upstream end of the air duct and facilitates heat transfer between the heat element 502 and the air filter 516. As used herein, “air curtain” describes a region of air moving at high velocity that acts as a barrier to prevent untreated air from moving therethrough (e.g., that blocks a flow of air moving longitudinally along the air passage 312). In another embodiment, the transverse air driver 513 may be coupled along a lower wall or a side wall of the air duct 501. In yet other embodiments, the TMIS 500 may include multiple transverse air drivers 513 configured to generate curtains of air at multiple positions along the air duct 501 (e.g., both upstream and downstream from the air filter 516). In at least one embodiment, the transverse air driver 513 is a transverse fan or blower.

As shown in FIG. 7, the transverse air driver 513 is arranged to direct the air curtain toward a lower, opposing wall of the air duct 501 and to reflect the air curtain off the lower wall so as to recirculate air (e.g., hot air) within a space between the transverse air driver 513 and the air filter 516, which can provide uniform heating of the air filter 316 and a more even distribution of heat across the cross-section of the air duct 301 (e.g., which can minimize temperature gradients within a section of the air duct in which the air filter 316 is located).

FIG. 8 and FIG. 9 show a TMIS 600 that includes a plurality of heat elements 602. The heat elements 602 are radiative heating elements. The radiative heating elements may be infrared (IR) heaters that emit IR light. The heat elements 602 may include a series of coils to generate the IR light. The heat elements 602 may be positioned in a linear array or column between the upper and lower walls of the air duct 601. In other embodiments, the heat elements 602 may be placed in corner regions of the air duct 601, which can improve temperature uniformity across the air filter 616. For example, the heat elements 602 can include elongated IR heaters with tubes extending diagonally between corners of the air duct 601 such as in an “X” shaped pattern (e.g., a pair of IR heating tubes with ends positioned in opposing corners of the air duct 601 and together forming an “X” shape when viewed along a flow direction through the air duct 601). In some embodiment, the IR heaters may include tubes with both hot and cold sections. For example, the tubes could include heated sections at the outer ends of the tube (e.g., proximate to the corner regions or sides of the air duct 601) with cold sections therebetween (e.g., a cold section at a middle section of the tube). In yet other embodiments, the IR heaters could include tubes with non-uniform heat flux along their length. For example, the tubes could be structured such that the heat flux increases between a middle section of the tube and the outer ends of the tube, to improve temperature uniformity across the air filter 616 (e.g., for the “X” pattern of tubes to prevent a substantial increase in heat flux where the tubes cross each other, etc.).

As shown in FIG. 8 and FIG. 9, the TMIS 600 may also include a plurality of separator elements 611, where each separator element 611 is disposed proximal a respective one of the heat elements 602 and extends along a length of the respective one of the heat elements 602. In at least one embodiment, the separator elements 611 are shields and/or reflectors made from metal or another suitable material that are positioned to redirect IR light from the heat elements 602 toward the air filter 616. The shields may be disposed between the heat elements 602 and the upstream or downstream end of the air duct 601 to reduce radiative heat loss to the upstream or downstream ends. The shields may form part of the IR heaters or be separate pieces from the IR heaters.

In some embodiments, the heat elements 602 and/or separator elements 611 may be fixedly coupled to the air duct 601 (e.g., stationary with respect to the air duct 601) and may be positioned to cover an entire surface of the air filter 616 in IR light. For example, the heat elements 602 and/or separator elements 611 may be staggered or otherwise arranged in an array at approximately equal intervals across the air passage 612, where each heat element 602 is directed toward a separate area or portion of the air filter 616. In other embodiments, and to improve temperature uniformity across the air filter 616 as described above, the heat elements 602 may be arranged in an “X” shaped pattern with ends of each heat element 602 disposed in a corner region of the air duct 616. In other embodiments, the heat elements 602 and/or separator elements 611 may be configured to oscillate (e.g., may include an oscillating mechanism or actuator, etc.) back and forth (e.g., up and down, side to side, etc.) to achieve uniform heating of the air filter 616. In some embodiments, the TMIS includes at least one IR heater on both sides of the air filter 616 and the IR heaters are positioned to fully irradiate both the upstream side and the downstream side of the air filter 316 (i.e., to irradiate all exposed sides and/or surfaces of the air filter 616), which can ensure more complete/uniform purification.

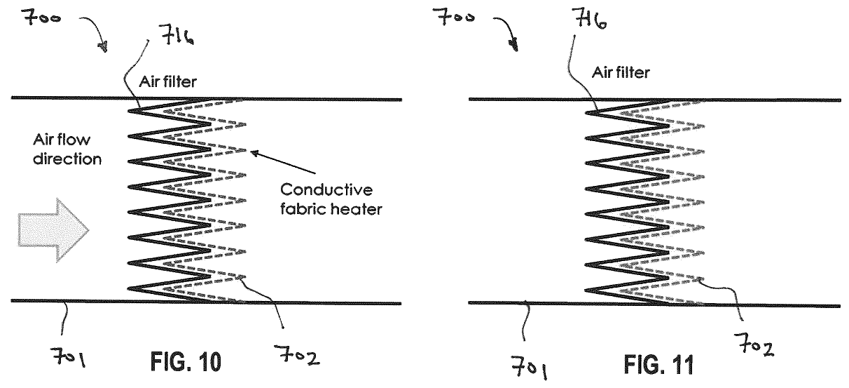

FIG. 10 and FIG. 11 show a TMIS 700 that includes a heat element 702 that is disposed within the air duct 701 and engages the air filter 716. The heat element 702 may include a resistance wire grid, a resistance mesh, a conductive fabric, or a combination thereof. In other embodiments, the heat element 702 may include another type of resistive or contact surface heater. As shown in FIG. 10 and FIG. 11, the heat element 702 is shaped to substantially match a shape of the air filter 716 so as to provide heating along substantially an entire length and/or surface of the air filter 716. For example, the heat element 702 may be shaped to conform to the pleats of the filter media (e.g., in an accordion shape or another shape that corresponds with the arrangement of the filter media). In some embodiments, the heat element 702 may be structured to facilitate filtration of air passing therethrough. For example, the heat element 702 may be a conductive heat fabric disposed on an upstream side of the air filter 716 and having a pore size that provides pre-filtering of dirt and debris passing through the air duct 701.

In at least one embodiment, the heat element 702 is separable from the air filter 716 and includes clips, magnets, or other fasteners to secure the heat element 702 to the air filter 716. In other embodiments, the heat element 702 may form part of the air filter 716 and may be sold with the air filter 716. In such an embodiment, the air filter 716 may include hook-ups (e.g., electrical connectors, etc.) that can be coupled to the microbial inactivation control system and used to power or otherwise control the heat element 702. In some embodiments, the heat element 702 comprises multiple separate heating panels that are engaged with the air filter 716. For example, the heat element 702 may include a first heating panel engaged with a first side (e.g., a downstream side) of the air filter 716 and a second heating panel engaged with a second side (e.g., an upstream side) of the air filter 716. The air filter 716 may be “sandwiched” or otherwise disposed between the first heating panel and the second heating panel to increase heat transfer to the filter media. The heating panels may be arranged to provide direct heating to all exposed sides and/or surfaces of the air filter.

In at least one embodiment, the heat element 702 is configured to facilitate inactivation of pathogens contained in the air filter 716 (e.g., to facilitate sanitization and/or purification of air passing therethrough). For example, the heat element 702 (e.g., heating panel) may be coated with an antimicrobial layer, antimicrobial agent, or otherwise treated with antimicrobials that inactivate viruses, bacteria, and other pathogens that come in contact with the heat element 702.

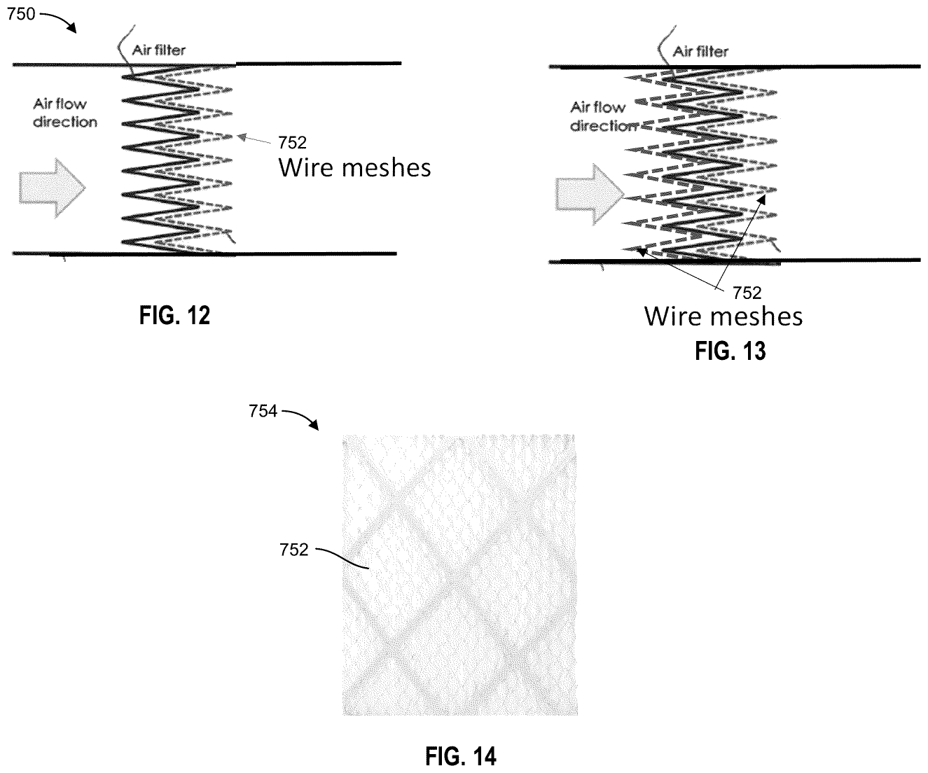

FIG. 12 shows a TMIS 750 that includes a heat element 752 that is made in the form of, or include, at least one wire mesh. The wire mesh has dual functions, including (1) providing heating to deactivate pathogens (e.g., via resistance wire heating, etc.), and (2) structurally supporting the filter media and filter pleats to prevent collapse of the filter media or filer pleats under loading (e.g., due to the pressure drop across the air filter during operation). The wire mesh can be applied on one side of the air filter (e.g., a downstream side of the air filter, as shown in FIG. 12) or the heat element 752 can include multiple wire meshes so as to support both sides of the air filter (e.g., a first wire mesh coupled to a downstream side of the air filter and a second wire mesh coupled to an upstream side of the air filter, as shown in FIG. 13). The wire mesh can be pleated, bent, or otherwise formed to match the shape and size of the pleats in the filter media. FIG. 14 shows a front view of an example air filter 754 that includes a wire mesh 752.

As described above, the thermal microbial inactivation systems of the present disclosure may include a microbial inactivation control system that is configured to control operation of the heat element, separator element, and/or air driver. In particular, the thermal microbial inactivation systems of the present disclosure may include a microbial inactivation control unit that is communicably coupled to the heat element, the separator element, and/or the air driver, and that is configured to coordinate operation of the heat element, the separator element, and/or air driver in response to a command for germicidal treatment (e.g., from a user interface of the microbial inactivation control unit, the IAQ controller, based on sensor data, etc.).

FIG. 3 shows a flow diagram of a method 800 of controlling a TMIS to inactivate viruses, bacteria, and other microbes and pathogens that are captured on an air filter, according to an illustrative embodiment. The TMIS may be any one of the TMISs described with reference to FIGS. 1-11 of the present application. In an operation 802, a microbial inactivation control unit queries a sensor and/or an HVAC control unit (e.g., the IAQ controller 104 of FIG. 1, etc.). For example, the microbial inactivation control unit may continuously or semi-continuously receive data from the air condition sensor and/or HVAC control unit via a communications or network interface. The sensor may be an air condition sensor such as the air condition sensor 328 of FIG. 2 and FIG. 3. The HVAC control unit may be a thermostat, a HVAC equipment controller, or another device configured to monitor and/or control HVAC equipment operation.

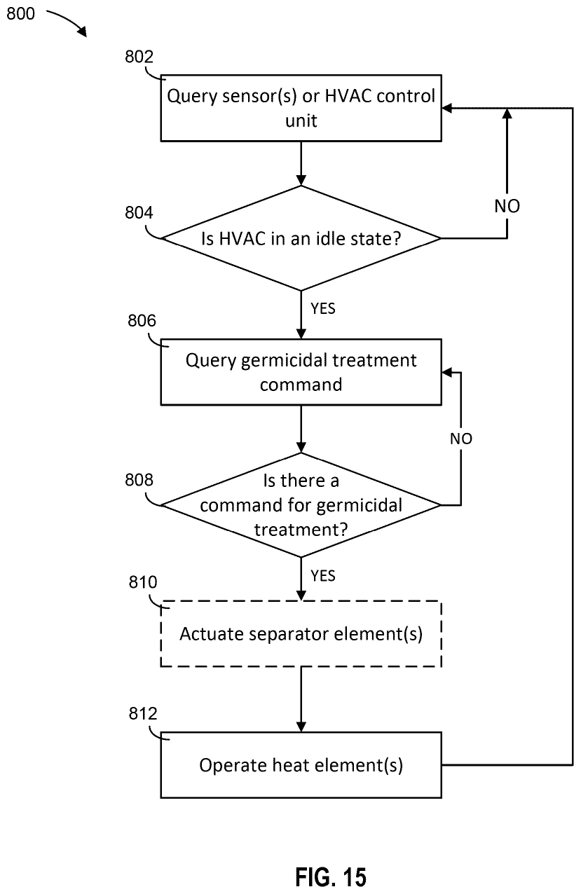

In an operation 804, the microbial inactivation control unit assesses the data from the sensor(s) and/or the HVAC control unit. The microbial inactivation control unit may assess the data to determine whether the HVAC system is in an idle state (e.g., an inactive state, an off-cycle state, etc.) or an active state (e.g., an on-cycle state, etc.). For example, the microbial inactivation control unit may compare the received sensor data to a value or range (e.g., an indicator value, etc.) stored in memory to determine whether the HVAC system is in an idle state. In at least one embodiment, as shown in FIG. 15, the microbial inactivation control unit may be configured to return to operation 802 if the HVAC system is in the active state.

In an operation 806, the microbial inactivation control unit queries for a germicidal treatment command in response to the data from the sensor and/or HVAC system indicating that the HVAC system is in an idle state. Operation 806 may include receiving, via the communications interface, a germicidal treatment command to perform germicidal treatment from a user interface of the microbial inactivation control unit or from the IAQ controller. The command may be triggered based on user input and/or based on a determination that a threshold period of time has elapsed between inactivation cycles.

In the event that the microbial inactivation control unit receives a germicidal treatment command (808=“YES”), the microbial inactivation control unit initiates an inactivation and/or sanitization cycle. The microbial inactivation control unit may be configured to coordinate operation of the heat element and the separator element to inactivate any pathogens captured by a filtration system. In an operation 810, the microbial inactivation control unit activates a separator element (e.g., a damper assembly, a transverse air driver, a shield or reflector element, etc.). For example, the microbial inactivation control unit may switch the separator element from a first mode of operation in which air is allowed to pass freely therethrough to a second mode of operation that at least partially blocks air flow through the separator element or across a flow stream produced by the separator element. In other embodiments, operation 810 may include switching an actuator for a shield or reflector element to a second mode of operation to redirect heat from the heat element toward an air filter. The microbial inactivation control unit may be configured to maintain the separator element in the second mode of operation until microbial treatment has completed.

In an operation 812, the microbial inactivation control unit activates a heat element (e.g., an infrared heater, a resistance wire grid, a resistance mesh, a conductive fabric, etc.) to increase the temperature of the air filter. Operation 812 may include maintaining activation of the heat element until data received from a temperature sensor (e.g., a temperature sensor proximal to the air filter or engaged with the air filter) indicates a threshold temperature or until the temperature sensor indicates that the filter element is within a treatment temperature range (e.g., within a range of temperatures that inactivates microbes and other pathogens). Operation 812 may include activating a fluid driver to increase heat transfer from the heat element to the air filter. In at least one embodiment, the microbial inactivation control unit is configured to maintain activation of the heat element and/or control the heat element to increase the temperature (e.g., average temperature as indicated by the temperature sensor) to a threshold temperature within a range between approximately 140° F. and 200° F. (or to any temperature within this treatment temperature range).

Operation 812 may further include controlling the heat element to maintain the temperature threshold for a microbial inactivation time period (e.g., a time period necessary to ensure that a threshold percentage of pathogens captured by the air filter have been inactivated, etc.). The microbial inactivation time period may be an experimentally determined time period that has been shown to sufficiently reduce pathogens below threshold levels. In at least one embodiment, the microbial inactivation time period may be within a range between approximately 15 seconds and 20 minutes. It should be appreciated that the microbial inactivation time period may differ for different threshold temperatures or temperature ranges.

In another embodiment, the microbial inactivation control unit is configured to operate the separator element and/or heat element based on a user-defined control algorithm (e.g., from a user interface, etc.). In a further embodiment, the microbial inactivation control unit is configured to operator the separator element and/or heat element using a control algorithm based on temperature data and/or other sensor measurements (e.g., based on data from the air condition sensor, etc.).

In at least one embodiment, the microbial inactivation control unit is configured to coordinate operation of the TMIS equipment (e.g., the heat element, the separator element, and/or the air driver) with the operation of the HVAC system. For example, the microbial inactivation control unit may control operation of the TMIS equipment using a control algorithm. The microbial inactivation control unit may be configured to determine operating parameters for the TMIS equipment based on a set of prioritization rules, IAQ and/or indoor comfort metrics. The operating parameters may include a general operating state of the TMIS and/or HVAC system (e.g., (e.g., HVAC system=“ON” and TMIS=“OFF”, HVAC system=“OFF” and TMIS=“ON”, or HVAC system=“ON” and TMIS=“ON”), or may include specific operating settings for at least one piece of HVAC or TMIS equipment.

Notwithstanding the embodiments described above in FIGS. 1-15, various modifications and inclusions to those embodiments are contemplated and considered within the scope of the present disclosure.

It is also to be understood that the construction and arrangement of the elements of the systems and methods as shown in the representative embodiments are illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter disclosed.

Accordingly, all such modifications are intended to be included within the scope of the present disclosure. Any means-plus-function clause is intended to cover the structures described herein as performing the recited function and not only structural equivalents but also equivalent structures. Other substitutions, modifications, changes, and omissions may be made in the design, operating conditions, and arrangement of the preferred and other illustrative embodiments without departing from scope of the present disclosure or from the scope of the appended claims.

Furthermore, functions and procedures described above may be performed by specialized equipment designed to perform the particular functions and procedures. The functions may also be performed by general-use equipment that executes commands related to the functions and procedures, or each function and procedure may be performed by a different piece of equipment with one piece of equipment serving as control or with a separate control device.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” “communicatively coupled,” or “operably coupled,” to each other to achieve the desired functionality, and any two components capable of being so associated can also be viewed as being “communicatively coupled” or “operably couplable,” to each other to achieve the desired functionality. Specific examples include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Similarly, unless otherwise specified, the phrase “based on” should not be construed in a limiting manner and thus should be understood as “based at least in part on.” Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances, where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Further, unless otherwise noted, the use of the words “approximate,” “about,” “around,” “substantially,” etc., mean plus or minus ten percent

Moreover, although the figures show a specific order of method operations, the order of the operations may differ from what is depicted. Also, two or more operations may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection operations, processing operations, comparison operations, and decision operations.

The various models, methods or processes outlined herein may be coded as software that is executable on one or more processors that employ any one of a variety of operating systems or platforms. Such models, methods, or processes may be executed on single local processors or may be executed across a plurality of remotely situated and networked processors (e.g., on the cloud). Additionally, such software may be written using any of a number of suitable programming languages and/or programming or scripting tools, and also may be compiled as executable machine language code or intermediate code that is executed on a framework or virtual machine.

In this respect, embodiments may provide a tangible, non-transitory computer readable storage medium (or multiple computer readable storage media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer-readable storage media) encoded with one or more programs that, when executed on one or more computers or other processors, perform methods that implement the various embodiments discussed above. The computer readable medium or media can be transportable, such that the program or programs stored thereon can be loaded onto one or more different computers or other processors to implement various aspects as discussed above. As used herein, the term “non-transitory computer-readable storage medium” encompasses only a computer-readable medium that can be considered to be an article of manufacture or a machine and excludes transitory signals.

The operations described in this specification can be implemented as operations performed by a data processing apparatus on data stored on one or more computer-readable storage devices or received from other sources. The term “data processing apparatus,” “processor,” or “computing device” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and an I/O device, e.g., a mouse or a touch sensitive screen, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some embodiments, a server transmits data (e.g., an HTJVIL page) to a client device (e.g., for purposes of displaying data to and receiving user input from a user interacting with the client device). Data generated at the client device (e.g., a result of the user interaction) can be received from the client device at the server.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. In some cases, the actions recited herein can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A system for thermal inactivation of pathogens, comprising:
an air duct configured to deliver air to an indoor space;
an air filter disposed within the air duct;
a heat element disposed within the air duct proximate the air filter, the heat element configured to heat the air filter and maintain the air filter at a threshold temperature for a microbial inactivation time period; and
a separator element disposed within the air duct proximate the heat element, the separator element configured to at least partially isolate the heat element and the air filter from an upstream end or a downstream end of the air duct.

2. The system of claim 1, wherein the separator element is operable between a first operating mode in which the separator element allows air to flow between the upstream end and the downstream end of the air duct, and a second operating mode in which the separator element at least partially blocks air from flowing between the upstream end and the downstream end of the air duct.

3. The system of claim 2, wherein the separator element is a first damper disposed in the air duct, the heat element disposed between the first damper and the air filter.

4. The system of claim 3, wherein the separator element further comprises a second damper disposed in the air duct on an opposite end of the air filter as the first damper.

5. The system of claim 2, further comprising an air driver configured to circulate air within a space between the heat element and the air filter when the separator element is in the second operating mode.

6. The system of claim 5, wherein the separator element and the air driver together comprise a single transverse air driver configured to produce an air curtain proximate the heat element or the air filter.

7. The system of claim 1, wherein the heat element is an infrared heater, and the separator element is a shield disposed between the infrared heater and the upstream end or the downstream end of the air duct.

8. The system of claim 1, wherein the heat element comprises a pair of infrared heaters arranged in an “X” shaped pattern within the air duct.

9. The system of claim 1, wherein the heat element is positioned to heat both an upstream side and a downstream side of the air filter.

10. The system of claim 1, wherein the threshold temperature is within a range between 140° F. and 200° F., inclusive, and wherein the microbial inactivation time period is within a range between 15 seconds and 20 minutes, inclusive.

11. The system of claim 1, further comprising a microbial inactivation control unit communicably coupled to the heat element and the separator element, the microbial inactivation control unit configured to coordinate operation of the heat element and the separator element in response to a command for germicidal treatment.

12. A system for thermal inactivation of pathogens, comprising:
an air duct configured to deliver air to an indoor space;
an air filter disposed within the air duct; and
a heat element disposed within the air duct and engaging the air filter, the heat element operable to selectively control a temperature of the air filter and maintain the air filter at a threshold temperature for a microbial inactivation period.

13. The system of claim 12, wherein the heat element is shaped to match a shape of the air filter so as to provide heating along substantially an entire length of the air filter.

14. The system of claim 12, wherein the heat element comprises at least one of a resistance wire grid, a resistance mesh, a wire mesh, or a conductive fabric.

15. The system of claim 12, wherein the heat element is treated with an antimicrobial agent.

16. The system of claim 12, wherein the heat element comprises a first heating panel engaged with an upstream side of the air filter, and a second heating panel engaged with a downstream side of the air filter.

17. A system for thermal inactivation of pathogens, comprising:
a microbial inactivation control unit having a communications interface that is configured to receive data from at least one of an air condition sensor or a heating, ventilation, and air conditioning (HVAC) control unit;
a heat element communicably coupled to the microbial inactivation control unit; and
a separator element communicably coupled to the microbial inactivation control unit, the separator element operable to selectively insulate the heat element from an environment surrounding the heat element, the microbial inactivation control unit configured to coordinate operation of the heat element and the separator element in response to the data indicating that an HVAC system is in an idle state.

18. The system of claim 17, further comprising a temperature sensor communicably coupled to the microbial inactivation control unit, wherein in response to the data indicating that the HVAC system is in the idle state the microbial inactivation control unit is configured to:
activate the separator element to switch the separator element from first mode of operation in which air is allowed to pass freely therethrough to a second mode of operation that at least partially blocks air flow through the separator element or across a flow stream produced by the separator element;
activate the heat element until the temperature sensor indicates a threshold temperature; and
control the heat element to maintain the threshold temperature for a microbial inactivation time period.

19. The system of claim 18, wherein the threshold temperature is within a range between 140° F. and 200° F., inclusive, and the microbial inactivation time period is within a range between 15 seconds and 20 minutes, inclusive.

20. The system of claim 18, wherein the separator element is one of a damper and a transverse fluid driver that is configured to produce a curtain of air.
